(19) United States
(12) Patent Application Publication
(10) Pub. No.: US 2015/0164450 A1
(43) Pub. Date: Jun. 18, 2015

(54) System and Method for Real Time 4D Quantification

(71) Applicant: Siemens Medical Solutions USA, Inc., Malvern, PA (US)

(72) Inventors: Balaji Rao Kotlapalli, Hoffman Estates, IL (US); Abdul Salim Vahabudeen, Algonquin, IL (US)

(21) Appl. No.: 14/546,519

(22) Filed: Nov. 18, 2014

Related U.S. Application Data
(60) Provisional application No. 61/917,412, filed on Dec. 18, 2013.

(51) Int. Cl. A61B 6/00; G06T 15/08

(52) U.S. Cl. CPC A61B 6/481; A61B 6/504; G06T 15/08; G06T 2207/30101

(57) ABSTRACT

A system and method include an interface to: receive imaging data representing a plurality of X-ray images in a patient volume including vessels during an acquisition period; one or more processors operative to: generate a 3D image of the patient volume showing a contrast medium flowing through the patient volume; and analyzing the patient volume during the acquisition period based on the flow of the contrast medium as the contrast medium flows through the patient volume to determine a stenosis level in one or more vessels; a display to display the 3D image of the patient volume as the contrast medium flows through the patient volume; and a memory. Numerous other aspects are provided.

### CROSS-REFERENCE TO RELATED APPLICATIONS

The present application claims priority to U.S. Provisional Patent Application No. 61/917,412, filed Dec. 18, 2013, entitled “REALTIME 4D QUANTIFICATION”, which is hereby incorporated in its entirety for all purposes.

### BACKGROUND

1. Field

The embodiments described below relate to the display and analysis of medical images acquired while contrast medium is present within a patient volume.

2. Description

According to conventional medical imaging, contrast medium is used to enhance the contrast of blood-carrying structures within patient anatomy. For example, contrast medium is introduced into a patient volume (e.g., via intravenous injection) and an image of the volume is acquired while the medium is located within the volume. In the image, structures which contain the medium appear darker than they would otherwise appear.

Conventional medical imaging systems have the ability to perform vessel analysis of 2D and 3D images after acquisition of the images. 2D images are X-ray images of a stationary region of interest acquired from a single plane, while 3D images are acquired by a rotational acquisition or by static acquisition of 2D images from more than one plane, and 3D reconstruction of the vessel of interest from the 2D images using 3D Reconstruction techniques. In these systems, a user manually selects a vessel of interest to be analyzed from an image, and computational analysis (e.g., a stenosis percentage) is not available during live image acquisition. As such, many physicians view the images directly, without computational analysis, to determine how to proceed with respect to the vessel.

Systems are desired which provide efficient presentation of 4D quantification results in real-time.

### BRIEF DESCRIPTION OF THE DRAWINGS

The construction and usage of embodiments will become readily apparent from consideration of the following specification as illustrated in the accompanying drawings, in which like reference numerals designate like parts, and wherein:

FIG. 1 illustrates a system according to some embodiments;

FIG. 2 is a flow diagram of a process according to some embodiments;

FIG. 3 illustrates a user interface according to some embodiments; and

FIG. 4 is a flow diagram of a process according to some embodiments.

### DETAILED DESCRIPTION

The following description is provided to enable any person in the art to make and use the described embodiments and sets forth the best mode contemplated for carrying out the described embodiments. Various modifications, however, will remain readily apparent to those in the art.

Those in the art will appreciate that various adaptations and modifications of the above-described embodiments can be configured without departing from the scope and spirit of the claims. Therefore, it is to be understood that the claims may be practiced other than as specifically described herein.

In conventional medical imaging applications, quantifiable analysis (e.g., via a computer) of the patient vessels is not available during live image acquisition. As such, many physicians determine the areas of stenosis (e.g., an abnormal narrowing of a patient vessel in the body) by viewing the images directly, and estimate the stenosis percentage in each area to then decide on the type of treatment to be performed to correct the stenosis.

Conventionally, to perform a 3D reconstruction of a vessel for a post-processing vessel analysis, the user, who may be a physician:

1. Acquires a biplane image (or two monoplane images in different angulations);

2. Calibrates the image;

3. Marks the vessel of interest on one plane of the scene by selecting multiple points on the vessel;

4. Marks the same vessel in the other plane by selecting multiple points on the vessel; and

5. Enables the 3D view to reconstruct the 3D view of the vessel as marked.

Systems and methods are disclosed for providing quantitative 4D vessel analysis (“4D quantification”) during live image acquisition. The 4D vessel analysis may be constructed and provided showing a visually-coded 3D view of vessels within a region of interest along a period of time, wherein time is the fourth dimension, in real-time (e.g., during live image acquisition). Based on the quantification, some embodiments of the invention may highlight the vessels with stenosis for display to a user. Some embodiments of the invention may provide 4D visualization of vessels within the region of interest, shown along with the 4D quantification results. 4D visualization represents a 3D view of the vessels, along with the time factor of flow which may be represented by different colors from the color spectrum, for example. This 4D view of the vessels of interest may be constructed during a live image (e.g., X-ray) acquisition.

One or more embodiments of the invention provide faster and improved quantitative vessel analysis and visualization of the results during interventional procedures as compared to conventional techniques. For example, one or more embodiments of the invention provide for: live vessel analysis, whereby the vessels may be automatically analyzed during the live image acquisition; 4D quantification, whereby quantitative 4D vessel analysis results may be displayed during the live image acquisition; and 4D vessel visualization within a region of interest (ROI), whereby the visualization of time indicating the flow of contrast medium through the 3D reconstructed vessel in the selected ROI is provided.

As used herein, a medical image study individually includes multiple image series of a patient anatomical portion and an image series in turn includes multiple image frames.

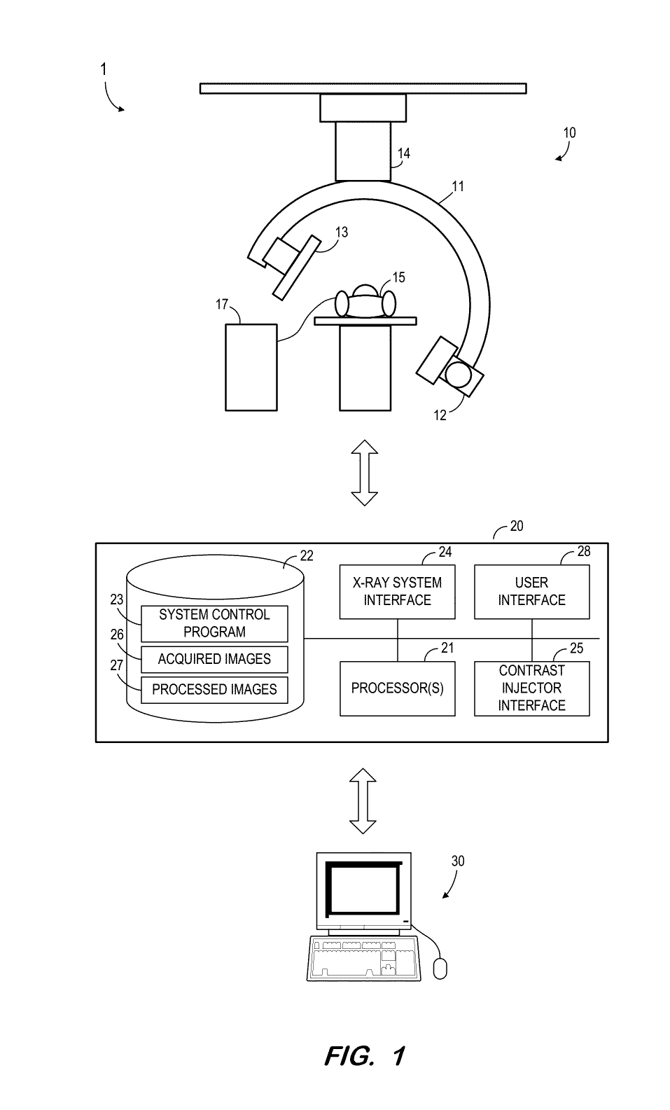

FIG. 1 illustrates a system 1 according to some embodiments. System 1 includes an image acquisition device, such as X-ray imaging system 10, for example. Other suitable image acquisition devices may be used (e.g., a magnetic resonance (MR) scanner, a CT scanner, an ultrasound device). The X-ray imaging system 10 may be referred to as a “scanner.” The system 1 also includes a control and processing system 20, and an operator terminal 30. Generally, according to some embodiments, the X-ray imaging system 10 introduces contrast medium into a patient volume and acquires X-ray images of the patient volume including vessels in the patient. The control and processing system 20 controls the X-ray imaging system 10 and receives the acquired images therefrom. The control and processing system 20 processes the images as described below and provides the processed images to a terminal 30 for display thereby. Such processing may be based on user input received by the terminal 30 and provided to the control and processing system 20 by the terminal 30. In some embodiments, a user may view each image produced as acquired by the X-ray imaging system 10 in real-time and additionally or alternatively may view images manufactured by the control and processing system 20 to show a 3D model reconstruction of the vessels in the patient.

X-ray imaging system 10 comprises a C-arm 11 on which a radiation source 12 and a radiation detector 13 are mounted. The C-arm 11 is mounted on a support 14 and is configured to translate clockwise or counter-clockwise with respect to the support 14. This translation rotates the radiation source 12 and the radiation detector 13 around a central volume while maintaining the physical relationship there between. Embodiments are not limited to C-arm-based imaging systems. A user may manipulate the C-arm 11 and instruct the X-ray imaging system 10 to capture, or acquire, images from different angles via a user interface (UI), for example. In some embodiments, the X-ray imaging system 10 may be in the shape of a tube. In some embodiments, the X-ray imaging system 10 may scan the anatomy of the patient in various planes, having more than one component to capture images from different angles, sometimes simultaneously.

The radiation source 12 may comprise any suitable radiation source, including but not limited to a Siemens Gigalix™ X-ray tube. In some embodiments, the radiation source 12 emits electron, photon, or other type of radiation having energies ranging from 50 to 150 keV.

The radiation detector 13 may comprise any system to acquire an image based on received X-ray radiation. The radiation detector 13 detects radiation provided by the radiation source 12 and passed through the patient anatomy. In some embodiments, the radiation detector 13 is a flat-panel imaging device using a scintillator layer and solid-state amorphous silicon photodiodes deployed in a two-dimensional array. The scintillator layer receives photons and generates light in proportion to the intensity of the received photons. The array of photodiodes receives the light and records the intensity of received light as stored electrical charge.

In other embodiments, the radiation detector 13 converts received photons to electrical charge without requiring a scintillator layer. The photons are absorbed directly by an array of amorphous selenium photoconductors. The photoconductors convert the photons directly to stored electrical charge. The radiation detector 13 may comprise a CCD or tube-based camera, including a light-proof housing within which are disposed a scintillator, a mirror, and a camera.

The charge developed and stored by the radiation detector 13 represents radiation intensities at each location of a radiation field produced by X-rays emitted from the radiation source 12. The radiation intensity at a particular location of the radiation field represents the attenuated properties of tissues lying along a divergent line between the radiation source 12 and the particular location of the radiation field.

A contrast medium injector 17 may comprise any known device or devices suitable to controllably introduce contrast medium into a patient volume. As described above, structures which contain contrast medium appear darker in X-ray images than they would otherwise appear. The contrast medium injector 17 may include a reservoir for each of one or more contrast media, and a patient interface such as medical-grade tubing terminating in a hollow needle.

The system 20 may comprise any general-purpose or dedicated computing system. Accordingly, the system 20 includes one or more processors 21 configured to execute processor-executable program code to cause the system 20 to operate as described herein, and a storage device 22 for storing the program code. The term “processor” as used herein is intended to include any processing device, such as, for example, one that includes a CPU (central processing unit) and/or other forms of processing circuitry. Further, the term “processor” may refer to more than one individual processor. The storage device 22 may comprise one or more fixed disks (e.g., hard drive), solid-state random access memory, and/or removable media (e.g., a thumb drive) mounted in a corresponding interface (e.g., a USB port).

Accordingly, computer software including instructions or code for performing the methodologies of the invention, as described herein, may be stored in one or more of the associated memory storage devices and, when ready to be utilized, loaded in part or in whole (for example, into random access memory) and implemented by a CPU. Such software could include, but is not limited to, firmware, resident software, microcode, and the like.

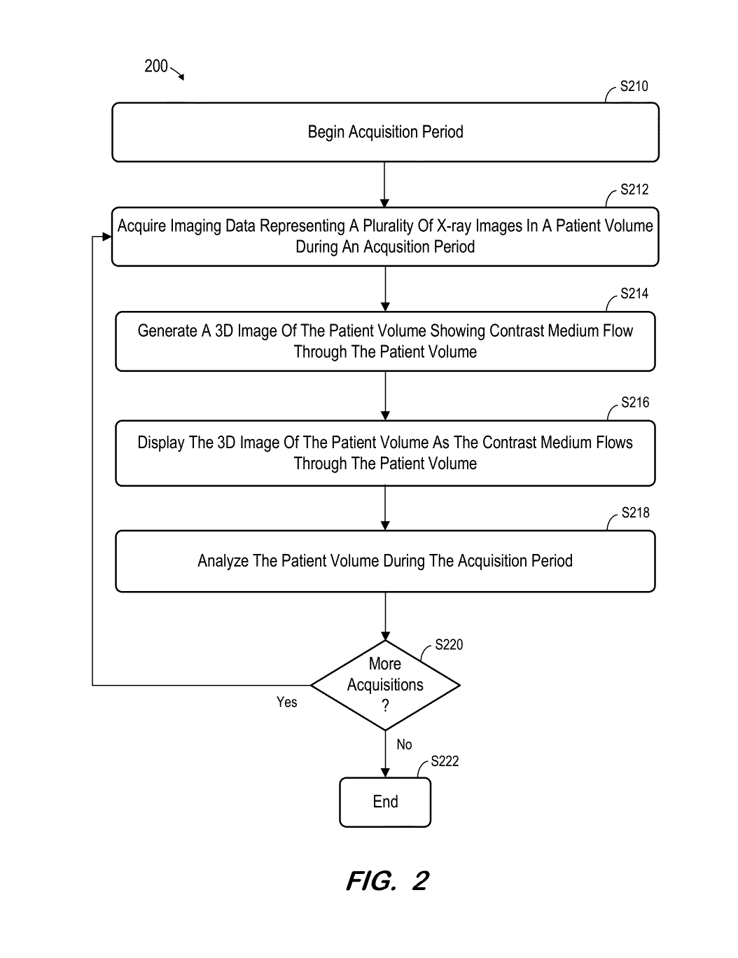

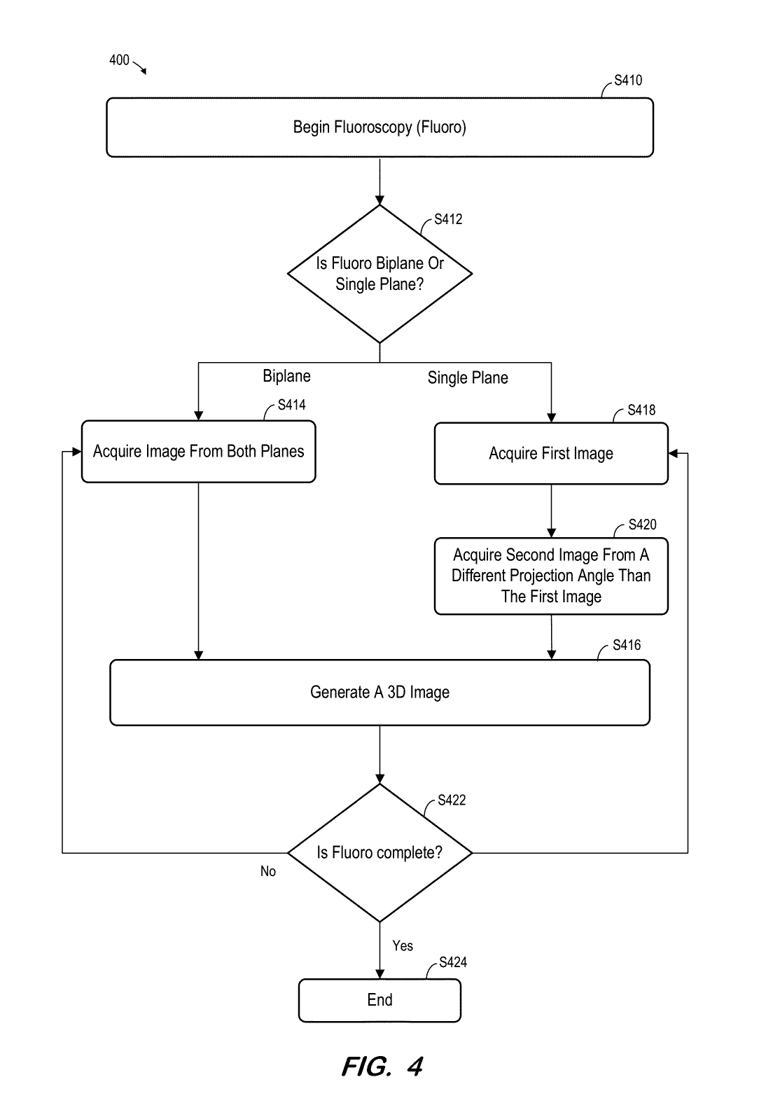

It is to be appreciated that at least a portion of the components shown in FIG. 1 may be implemented in various forms of hardware, software or combinations thereof, e.g., one or more digital signal processors with associated memory, application-specific integrated circuits, functional circuitry, etc. It should also be noted that some or all of the system 20, for example, can be incorporated in an application specific or general-use integrated circuit. For example, one or more method steps described below with respect to FIGS. 2 and 4, or elsewhere, could be implemented in hardware in an application specific integrated circuit (ASIC) rather than using software.

The storage device 22 stores program code of a system control program 23. One or more processors 21 may execute the system control program 23 to move the C-arm 14, to cause the radiation source 12 to emit radiation, to control the detector 13 to acquire an image, to cause the injector 17 to introduce contrast medium into a volume of a patient 15, and to perform any other function. In some embodiments, these functions may be performed in response to user commands. In this regard, the system 20 includes an X-ray system interface 24 and a contrast injector medium interface 25 for communication with the system 10.

Images acquired from the system 10 are stored in the data storage device/memory 22 as acquired images 26, in DICOM or another data format. Each acquired image 26 may be further associated with details of its acquisition, including but not limited to imaging plane position and angle, imaging position, radiation source-to-detector distance, patient anatomy imaged, patient position, contrast medium bolus injection profile, X-ray tube voltage, image resolution and radiation dosage. In one or more embodiments, first and second sets of data images are stored in the data storage device 22. The first and second sets of data images may be taken at different stages of a treatment procedure and represent corresponding first and second image sequences individually comprising multiple temporally sequential individual image frames of, for example, vessels or other portions of a patient anatomy. The sequential individual images may encompass introduction of a contrast medium into patient vessels.

Processor(s) 21 may execute the system control program 23 to process acquired images 26, resulting in one or more processed images 27. Processed images 27 may be provided to the terminal 30 via a user interface 28 of system 20. The user interface 28 may also receive input from the terminal 30, which is used to control processing of acquired images 26 as described below.

The terminal 30 may simply comprise a display device and an input device coupled to the system 20. The terminal 30 displays processed images 27 received from the system 20 and receives user input which may be transmitted to the system 20 and used thereby to generate new processed images 27 for subsequent display by the terminal 30. In some embodiments the terminal 30 is a separate computing device such as, but not limited to, a desktop computer, a lap-top computer, a tablet computer, and a smartphone. In some embodiments, the input device may be a keyboard, mouse, touchscreen, voice data entry and interpretation device, or a foot pedal. In some embodiments, the foot pedal provides the user with an alternative means to operate the system 20 during diagnosis or treatment of a patient when the user's (e.g., physician) hands may be preoccupied with surgeon devices, catheters, other medical instruments, or other user interface control devices, for example.

Each of the system 10, the system 20 and the terminal 30 may include other elements which are necessary for the operation thereof, as well as additional elements for providing functions other than those described herein.

According to the illustrated embodiment, the control and processing system 20 controls the elements of the X-ray imaging system 10. The control and processing system 20 also processes images received from the X-ray imaging system 10. Moreover, the system 20 receives input from the terminal 30 and provides processed images to the terminal 30. In one or more embodiments, images are generated in response to predetermined user specific preferences. Embodiments are not limited to a single system performing each of these functions. For example, X-ray imaging system 10 may be controlled by a dedicated control system, with the acquired images being provided to a separate imaging processing system over a computer network or via a physical storage medium (e.g., a DVD).

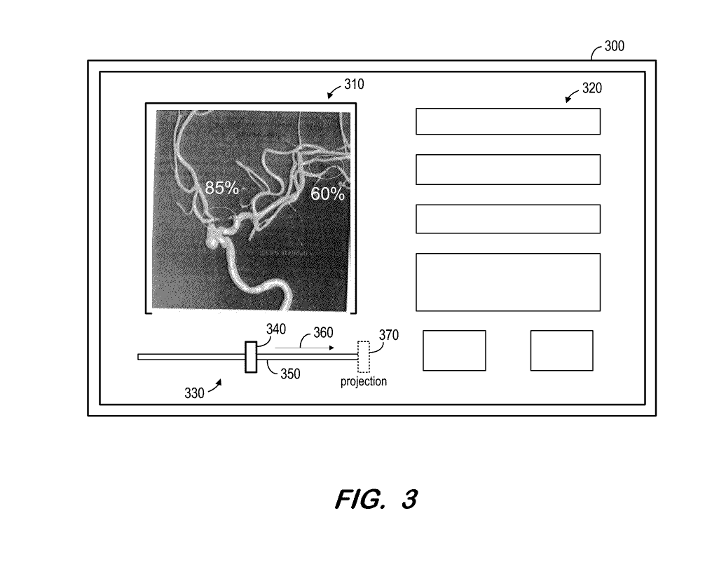

Turning to FIGS. 2-3, in one example of operation according to some embodiments, FIG. 2 is a flow diagram of a process 200 according to some embodiments. Process 200 and other processes described herein may be performed using any suitable combination of hardware (e.g., circuit(s)), software or manual means. In one or more embodiments, the control and processing system 20 is conditioned to perform the process 200, such that the processing system 20 is a special-purpose element configured to perform operations not performable by a general-purpose computer or device. Software embodying these processes may be stored by any non-transitory tangible medium including a fixed disk, a floppy disk, a CD, a DVD, a Flash drive, or a magnetic tape. Examples of these processes will be described below with respect to the elements of system 20, but embodiments are not limited thereto.

Initially, at S210 an acquisition period begins. In one or more embodiments, the beginning of the acquisition period may be user defined. For example, the user may define the acquisition period to begin at a particular time scheduled at the user interface 28, or may step on a foot pedal in the examination room to begin the acquisition period. The processor 21 receives image data representing a plurality of X-ray images of a patient volume in S212. In one or more embodiments, the acquisition/receipt of images occurs while contrast medium is injected into the vessels of the patient. In one or more embodiments, the user (e.g., physician) may directly inject the contrast medium into the vessels of the patient and in other embodiments, the system 20 and contrast medium injector interface 25 control the injection. In one or more embodiments, the processor 21 requests to receive a frame image(s) of the patient vessel. If the system is a biplane system, the processor 21 requests to receive a frame image of the patient vessel from both planes. If the system is a mono-plane system, the processor 21 requests to receive a frame image of the patient vessel from a first plane, and then from a second plane at a different angulation from the first plane. If the system includes a C-arm rotating about the patient, the processor 21 requests to receive a plurality of frame images of the patient vessel from a plurality of planes as the images are captured while the C-arm rotates about the patient.

Next, in S214, a 3D image or model of the patient volume showing a contrast medium flowing through the patient volume is generated or reconstructed by the processor 21 based on the imaging data received at S212. The contrast medium allows the vessels to stand out from the surrounding anatomy in the imaging data. As the contrast medium flows through the vessels, the flow is visualized on the 3D reconstructed model built by the processor 21 in S214. For example, in one or more embodiments, the flow of contrast medium is indicated by at least one visual attribute (e.g., color) added to the 3D image. In one or more embodiments, the visual attribute indicates blood flow transit time. In S216, the 3D image of the patient volume depicting the flow of contrast medium over time is displayed at terminal 30, and enables the user (e.g., physician) to see a 4D view of the vessels.

A vessel analysis is performed during the acquisition period by the processor 21 at S218 based on the flow of contrast medium depicted in the 3D image. For example, in a 15 frames per second (fps) image acquisition, the vessel analysis may occur at S218 in less than 66 ms to avoid frame loss. In one or more embodiments, the vessel analysis (i.e., 4D quantification) includes an assessment of vessel area(s) and a determination, based on the 3D image, of the amount (e.g., percentage) of stenosis in that area(s) at a particular time compared to a non-narrowed vessel. In one or more embodiments, a vessel may be rated as having a high level of stenosis, a medium level of stenosis, or a low level of stenosis based on a comparison of the percentage of stenosis to threshold values. For example, a rating greater than 70% may be a high level of stenosis, while rating less than 30% may be a low level, and a rating between and including 30% and 70% may be a medium level. In one or more embodiments, the analysis may be performed by any suitable method. For example, the method may be one of Quantitative Coronary Analysis or Quantitative Vascular Analysis, both provided by Pie Medical Imaging of The Netherlands, or the Warfarin-Aspirin Symptomatic Intracranial Disease method. In one or more embodiments the type of vessel analysis used may depend on the organ being diagnosed.

In one or more embodiments, vessels having a high level of stenosis are displayed to the user. In one or more embodiments, the user determines which levels of stenosis-afflicted vessels are displayed. In addition to the level and percentage of stenosis determined as part of the 4D quantification, the analysis may also include a determination of at least one of: a length of the portion of the vessel where the stenosis is present; a minimal luminal diameter of the vessel with stenosis (which may include a percentage of the stenosis); and a time for the contrast medium to flow across the vessel with stenosis. In one or more embodiments, the user determines whether 4D quantification is performed for one or more of those vessels labeled as having a high level of stenosis, medium level of stenosis, or low level of stenosis. In one or more embodiments, the 4D quantification may be performed for stenosis levels selected by the user prior to the beginning of the acquisition period. In one or more embodiments, the 4D quantification may be performed for stenosis levels selected by the user during the acquisition period.

Then in S220, it is determined whether more images should be acquired to refine the 3D image of the vessel generated in S214. In one or more embodiments, one or more factors may be used to determine whether more acquisitions are required. In one or more embodiments, the determination may be made per case. For example, where the physician is performing diagnosis, it may be desirable to acquire more images to see the complete vessel anatomy. As another example, where the vessels are not clearly seen, the physician may change the angle of the detector to see a better view of the vessels, and therefore acquire more images. As yet another example, the physician may acquire more images to see the effectiveness of treatment. In one or more embodiments, another dose of contrast medium may be applied during reception of the new images at S212. In one or more embodiments, this determination is made by one of the user or the processor 21. For example, the user may maintain the foot pedal in the down position, thereby providing an instruction to acquire more images. If it is determined that more images should be acquired, the process returns to S212. If not, the process 200 and acquisition period ends in S222.

FIG. 3 illustrates interface 300 for displaying the 3D image of the patient vessel and the analysis results according to some embodiments. Interface 300 may be displayed by a display device of terminal 30 in response to execution of program code of the system control program 23 by processor(s) 21.

Interface 300 includes area 310 for displaying the 3D image depicting contrast medium flow over time. The 3D image also displays the results of the 4D quantification described above with respect to S218. For example, area 310 of FIG. 3 displays a vessel view with two stenosis locations circled in white, and a percentage or level of stenosis listed for each location during the acquisition period.

Interface 300 also includes areas 320 for displaying any suitable information that is or becomes known. For example, the area 320 may present one or more image acquisition parameters (e.g., time interval, sub-sampling, etc.), patient biometric data, or the like. Areas 320 may also display results of the 4D quantification such as, but not limited to, a length of the portion of a selected vessel where the stenosis is present; a minimal luminal diameter of the vessel with stenosis (which may, in some embodiments, include a percentage of the stenosis); and a time required for the contrast medium to flow across the vessel with stenosis. Areas 320 may also or alternatively display information relating to images displayed in area 310, such as, but not limited to, histograms, filtering parameters, and biological information determined based on the images.

Areas 320 may also or alternatively include one or more user interface controls. These controls may allow an operator/user to change the information displayed by the interface 300 or to control image processing performed by system 20, or to perform any other suitable operation.

In this regard, an operator/user operates control 330 in some embodiments to select the region of interest (ROI) that the user would like to view, as well as the information associated with that ROI. As used herein, ROI refers to the area of the vessel with stenosis. In some embodiments, the processor 21 may automatically choose the ROI based on where stenosis is shown in a vessel. In some embodiments, multiple controls 330 are provided. Control 330 consists of slider 340 and slider bar 350. An operator may move slider 340 along slider bar 350 as illustrated by arrow 360 to position 370 using a touch screen, a mouse, a keyboard or any other input device. The position of the slider 340 may be associated with a particular projection angle of the vessel to display the ROI per user input.

Embodiments are not limited to the content and arrangement discussed above with respect to FIG. 3. Any one or more user interface controls may be used to change the displayed and analyzed vessel discussed herein. Such controls include, but are not limited to, next input boxes, dials, gestures, etc. Moreover, in some embodiments, an operator may transmit a selected image showing a ROI to the system 20 via an input device (e.g., joystick, mouse, touchpad) without the use of any visually manipulable user interface controls. In such embodiments, the only visual feedback of the thusly-changed projection angle may be the resulting changes in the displayed images.

Turning to FIG. 4, in one example of operation, FIG. 4 is a flow diagram of a process 400 to acquire imaging data and generate a 3D image as described above with respect to S212 and S214, according to some embodiments. As described above, process 400 and other processes described herein may be performed using any suitable combination of hardware (e.g., circuit(s)), software or manual means. In one or more embodiments, the control and processing system 20 is conditioned to perform the process 400, such that the processing system 20 is a special-purpose element configured to perform operations not performable by a general-purpose computer or device.

Initially, fluoroscopy (fluoro) begins at S410. Fluoroscopy is an imaging technique that uses X-rays to obtain real-time moving images of the internal structures of a patient through the use of a fluoroscope (e.g., an X-ray source and an X-ray detector between which a patient is placed.) Conventionally, a fluoro may be performed prior to an acquisition period, as described above with respect to FIG. 2. A fluoro may be performed with a low dose X-ray that does not show as much detail as an acquisition described above with respect to FIG. 2, but may be used to determine whether a patient has a vessel that may have stenosis, while exposing a patient to limited radiation. In one or more embodiments, the starting time of the fluoro may be user defined. For example, the user may define the fluoro to begin at a particular time scheduled at the user interface 28, or may step on a foot pedal in the examination room to begin the fluoro.

In S412, a determination is made as to whether the fluoro is a biplane fluoro or a single plane fluoro. To reconstruct a 3D model, images from at least two different projection angles may be used. In a biplane fluoro, two different C-arms may be used to each acquire an image from a different angle at substantially the same time, such that two images are acquired per acquisition. In a mono- or single-plane fluoro, one C-arm is used to acquire a first image, and then the C-arm is moved to a different projection angle to acquire a second image, such that one image is acquired per acquisition. If S412 indicates the fluoro is a biplane, the process 400 proceeds to S414, and an image is acquired from both planes at substantially the same time, in one or more embodiments. The processor 21 then reconstructs or generates a 3D image from the frame images of both planes in S416, in one or more embodiments. In one or more embodiments, for example, in a 30 frame per second (fps) fluoro, the 3D reconstruction may occur in under 33 ms so as to avoid frame loss.

The process 400 then proceeds to S422 and it is determined whether the fluoro is complete. If step S422 indicates the fluoro is not complete, flow returns to step S414. In one or more embodiments, this determination is made by one of the user or the processor 21. For example, the user may maintain the foot pedal in the down position, thereby instructing processor 21 to return to S414 to acquire more images. In one or more embodiments, the last-generated 3D image may be displayed while the processor 21 is receiving more fluoros. Then, as the next frames of the biplane fluoro are received, the 3D image displayed to the user is updated based on the latest 3D image constructed by the processor 21. If S422 indicates the fluoro is complete, the process proceeds to S424, and the process 400 ends.

If S412 indicates the fluoro is a monoplane fluoro, the process 400 proceeds to S418, and an image associated with a first projection angle is acquired. Then in S420, an image associated with a second projection angle is acquired. The process then proceeds to S416, and the processor 21 constructs or generates a 3D image from the first and second images. According to some embodiments, images may be acquired from more than two projection angles and used to generate the 3D image at S416.

In one or more embodiments, during acquisition of the image associated with the second projection angle, the processor 21 may create a reference image from the image in the first plane and the image from the second projection angel or plane for use as reference for the 3D image reconstruction in S416, while the subsequent fluoros are performed at a different angulation. The 3D image may be reconstructed by the processor 21 and displayed on terminal 30 based on the current frame of the current fluoro and the reference frame from the previous fluoro, such that the 3D image is refined as subsequent fluoros are acquired. After 3D image generation in S416, the process proceeds to S422 and it is determined whether the fluoro is complete. If S422 indicates the fluoro is not complete, the process returns to S418. If S422 indicates the fluoro is complete, the process proceeds to 424, and the process 400 ends.

At the end of the fluoros, a 3D image of the complete vessel structure may be displayed on the terminal 30. In one or more embodiments, a user may review the 3D image from the fluoro to determine whether to perform the process 200 described above with respect to FIG. 2. In one or more embodiments, if stenosis is present in the 3D image of the vessel provided via the fluoro, the process 200 is performed.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

One or more embodiments of the invention, or elements thereof, can be implemented in the form of an apparatus including a memory and at least one processor that is coupled to the memory and operative to perform exemplary method steps.

As noted, aspects of the present invention may take the form of a computer program product embodied in one or more computer readable medium(s) having computer readable program code embodied thereon. Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. Some specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Aspects of the present invention are described herein with reference to flowchart illustrations and/or diagrams of methods, apparatus (systems) and computer program products according to embodiments of the invention. It will be understood that each block of the flowchart illustrations and/or diagrams, and combinations of blocks in the flowchart illustrations and/or diagrams, can be implemented by computer program instructions. These computer program instructions may be provided to a processor of a general purpose computer, special purpose computer, or other programmable data processing apparatus to produce a special purpose machine, such that the instructions, which execute via the processor of the computer or other programmable data processing apparatus, create means for implementing the functions/acts specified in the flowchart and/or diagram block or blocks.

The flowchart and block diagrams in the Figures illustrate the architecture, functionality, and operation of possible implementations of systems, methods and computer program products according to various embodiments of the present invention. In this regard, each block in the flowchart or diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the diagrams and/or flowchart illustration, and combinations of blocks in the diagrams and/or flowchart illustration, can be implemented by special purpose hardware-based systems that perform the specified functions or acts, or combinations of special purpose hardware and computer instructions.

It should be noted that any of the methods described herein can include an additional step of providing a system comprising distinct software modules embodied on a computer readable storage medium; the modules can include, for example, any or all of the elements depicted in the block diagrams and/or described herein; by way of example and not limitation, an imaging module, and a stenosis level module. The method steps can then be carried out using the distinct software modules and/or sub-modules of the system, as described above, executing on one or more hardware processors 21. Further, a computer program product can include a computer-readable storage medium with code adapted to be implemented to carry out one or more method steps described herein, including the provision of the system with the distinct software modules.

In any case, it should be understood that the components illustrated herein may be implemented in various forms of hardware, software, or combinations thereof; for example, application specific integrated circuit(s) (ASICS), functional circuitry, one or more appropriately programmed general purpose digital computers with associated memory (thereby forming special purpose computers), and the like. Given the teachings of the invention provided herein, one of ordinary skill in the related art will be able to contemplate other implementations of the components of the invention

Those in the art will appreciate that various adaptations and modifications of the above-described embodiments can be configured without departing from the scope and spirit of the claims. Therefore, it is to be understood that the claims may be practiced other than as specifically described herein.

What is claimed is:

1. A method comprising:
receiving imaging data representing a plurality of X-ray images in a patient volume including vessels during an acquisition period;
generating, during the acquisition period, a 3D image of the patient volume depicting a contrast medium flow through the patient volume;
displaying, during the acquisition period, the 3D image of the patient volume depicting the contrast medium flow through the patient volume; and
analyzing the patient volume during the acquisition period based on the flow of the contrast medium to determine a stenosis level in one or more of the vessels.

2. The method of claim 1, further comprising:
displaying a stenosis level on the 3D image during the acquisition period.

3. The method of claim 1, wherein the acquisition period is user defined.

4. The method of claim 1, further comprising:
acquiring one or more new X-ray images in addition to the plurality of X-ray images; and
generating a refined 3D image based on the newly acquired X-ray images and the 3D image.

5. The method of claim 1, wherein the stenosis level is one of: high, medium and low.

6. The method of claim 1, wherein determining a stenosis level in one or more vessels further comprises:
determining a percentage of stenosis compared to a non-narrowed vessel; and
comparing the percentage of stenosis to a threshold value.

7. The method of claim 5, wherein vessels having a high stenosis level are indicated.

8. The method of claim 1, further comprising:
determining a presence of stenosis in one or more vessels.

9. The method of claim 8, wherein analyzing the patient volume further comprises at least one of:
determining a length of a portion of the vessel where stenosis is present;
determining a minimal luminal diameter of the vessel where stenosis is present;
determining a time for the contrast medium to flow across the vessel where stenosis is present.

10. The method of claim 9, wherein the minimal luminal diameter of the vessel where stenosis is present includes the percentage of the stenosis.

11. The method of claim 1, further comprising providing a system, wherein the system comprises distinct software modules, each of the distinct software modules being embodied on a computer-readable storage medium, and wherein the distinct software modules comprise an imaging module, and a stenosis level module;
wherein:
the generation of the 3D image of the patient volume showing the contrast medium flowing through the patient volume is carried out by the imaging module executing on at least one hardware processor; and
the analysis of the patient volume based on the flow of contrast medium is carried out by the stenosis level module executing on the at least one hardware processor.

12. A system comprising:
an interface to:
receive imaging data representing a plurality of X-ray images in a patient volume including vessels during an acquisition period;
one or more processors operative to:
generate a 3D image of the patient volume showing a contrast medium flowing through the patient volume;
analyzing the patient volume during the acquisition period based on the flow of the contrast medium as the contrast medium flows through the patient volume to determine a stenosis level in one or more vessels;
a display to display the 3D image of the patient volume as the contrast medium flows through the patient volume; and
a memory, coupled to the one or more processors, to store image data and to store program instructions for execution by the one or more processors.

13. The system of claim 12, wherein the display includes patient volumes having the stenosis level greater than a threshold value.

14. The system of claim 12, wherein the flow of contrast medium is indicated by at least one visual attribute.

15. The system of claim 12, wherein the visual attribute indicates blood flow transit time information.

16. The system of claim 12, wherein the imaging data is received during an acquisition period, and wherein the analysis occurs during the acquisition period.

17. The system of claim 12 wherein the analysis of the patient volume includes at least one of:
a determination of a length of a portion of the vessel where stenosis is present;
a determination of a minimal luminal diameter of the vessel where stenosis is present; and
a determination of a time for the contrast medium to flow across the vessel where stenosis is present.

18. The system of claim 12, further comprising a plurality of distinct software modules, each of the distinct software module being embodied on a computer-readable storage medium, and wherein the distinct software modules comprise an imaging module, and a stenosis level module;
wherein:
the at least one processor is operative to analyzing the patient volume based on the flow of the contrast medium as the contrast medium flows through the patient volume to determine a stenosis level in one or more vessels by executing the stenosis level module.
